(19) United States
(12) Patent Application Publication
(10) Pub. No.: US 2025/0099228 A1
(43) Pub. Date: Mar. 27, 2025

(54) MEMBER FOR INTRAOCULAR INSERTION AND METHOD FOR INSERTING MEMBER FOR INTRAOCULAR INSERTION

(71) Applicant: WELLC LLC, Busan (KR)

(72) Inventor: Ki Sung PARK, Hwaseong-si (KR)

(73) Assignee: WELLC LLC, Busan (KR)

(21) Appl. No.: 18/563,609

(22) PCT Filed: Sep. 6, 2022
(86) PCT No.: PCT/KR2022/013394

§ 371 Date: Nov. 22, 2023

(30) Foreign Application Priority Data
May 18, 2022 (KR) 10-2022-0060908

(51) Int. Cl. A61F 2/14

(52) U.S. Cl. CPC A61F 2/147

(57) ABSTRACT

A member for intraocular insertion and method for inserting a member for intraocular insertion is provided. The member for intraocular insertion comprises a ring assembly enclosing a portion of the cornea and comprising a plurality of rings having the same center, wherein the plurality of rings have the shape of concentric circles with different sizes, and a plurality of connection members for connecting between the plurality of rings, wherein each of the plurality of connection members is disposed apart from an adjacent connection member by the same angle with respect to the center.

### TECHNICAL FIELD

The present invention relates to a member for intraocular insertion and a method for inserting the member for intraocular insertion.

### BACKGROUND ART

Since the cornea has a gel-type material, ectasia due to intraocular pressure occurs in the thin portion of the cornea. In this case, the dilated portion creates a curvature deviation, which causes abnormal vision and also causes a tear film defect, thereby resulting in xerophthalmia. If corneal ectasia due to intraocular pressure continues, eventually, a situation in which the cornea is perforated may occur.

The conventional corneal collagen cross-linking used to prevent corneal deformation is a medical technology that inhibits and stabilizes the progression of keratoconus by applying vitamin B2 to the corneal stroma and then exposing it to ultraviolet light to strengthen the cross-linking between collagen fibers, thereby increasing the strength of the cornea.

Such corneal collagen cross-linking has disadvantages in that cross-linking is not made quantitatively and uniformly, and vision becomes blurred after the procedure, and as time goes by, the bonding force decreases, which may lead to loss of effectiveness.

### SUMMARY

Aspects of the present invention to be solved to provide a member for intraocular insertion.

Aspects of the present invention to be solved to provide a method for inserting the member for intraocular insertion.

According to some aspects of the present disclosure, a member for intraocular insertion comprises a ring assembly enclosing a portion of the cornea and comprising a plurality of rings having the same center, wherein the plurality of rings have the shape of concentric circles with different sizes, and a plurality of connection members for connecting between the plurality of rings, wherein each of the plurality of connection members is disposed apart from an adjacent connection member by the same angle with respect to the center.

According to some aspects, the member for intraocular insertion further comprises an auxiliary connection member for connecting between the plurality of rings and disposed where the connection members are not disposed.

According to some aspects, the material of the ring is at least one of polymethylmethacrylate (PMMA), gold, alloy; platinum, titanium, and stainless steel.

According to some aspects of the present disclosure, a member for intraocular insertion comprises a ring assembly enclosing a portion of the cornea and comprising a plurality of rings having the same center, wherein the plurality of rings have the shape of ellipses with the same eccentricity and different sizes, and a plurality of connection members for connecting between the plurality of rings, wherein each of the plurality of connection members is disposed apart from an adjacent connection member by the same angle with respect to the center.

According to some aspects, the member for intraocular insertion further comprises an auxiliary connection member for connecting between the plurality of rings and disposed where the connection members are not disposed.

According to some aspects, the material of the ring is at least one of polymethylmethacry late, gold, alloy, platinum, titanium, and stainless steel.

According to some aspects of the present disclosure, a method for inserting a member for intraocular insertion, comprises the steps of selecting a member for intraocular insertion to be inserted into a user's cornea, wherein the member for intraocular insertion comprises a ring assembly enclosing a portion of the cornea and comprising a plurality of rings having the same center, wherein the plurality of concentric rings have the shape of concentric circles with different sizes, and a plurality of connection members for connecting between the plurality of rings, wherein each of the plurality of connection members is disposed apart from an adjacent connection member by the same angle with respect to the center, incising a portion of the cornea to create an incised portion and inserting the selected member for intraocular insertion into the incised portion.

According to some aspects, the step of selecting a member for intraocular insertion to be inserted into a user's cornea comprises the step of identifying the corneal state comprising a curvature of the incised portion into which the member for intraocular insertion is inserted, and the step of selecting a member for intraocular insertion corresponding to the identified corneal state from a first set of members for intraocular insertion.

According to some aspects, each member for intraocular insertion of the first set of members for intraocular insertion is different from each other in at least one of the curvature of each member for intraocular insertion, the distance from the center to the smallest ring among the plurality of rings, the distance from the center to the largest ring among the plurality of rings, the intervals between the plurality of rings, whether the intervals between the plurality of rings are constant or not, and whether the auxiliary connection members are disposed where the connection members are not disposed or not.

According to some aspects, the step of incising a portion of the cornea to create an incised portion comprises the step of creating a groove having the same shape as that of the selected member for intraocular insertion in at least one site of corneal epithelium and Bowman's membrane among constitutive tissues of the cornea, or the step of creating a groove having a toric shape with a predetermined size in the at least one site.

According to some aspects, the step of incising a portion of the cornea to create an incised portion comprises the step of incising the corneal stroma among constitutive tissues of the cornea in a horizontal straight line to form an upper corneal stroma and a lower corneal stroma.

According to some aspects, the step of inserting the selected member for intraocular insertion into the incised portion comprises the step of inserting the member for intraocular insertion between the upper corneal stroma and the lower corneal stroma.

According to some aspects, the step of incising the corneal stroma in a horizontal straight line further comprises the step of creating a groove having the same shape as that of the selected member for intraocular insertion in the lower corneal stroma, or the step of creating a groove having a toric shape with a predetermined size in the lower corneal stroma.

According to some aspects, the method for inserting a member for intraocular insertion further comprises the step of suturing between the upper corneal stroma and the lower corneal stroma after the selected member for intraocular insertion is inserted into the incised portion.

According to some aspects, each of the plurality of connection members is disposed apart from an adjacent connection member by the same angle with respect to the center.

According to some aspects, the member for intraocular insertion further comprises an auxiliary connection member for connecting between the plurality of rings and disposed where the connection members are not disposed.

According to some aspects, the material of the ring is at least one of polymethylmethacry late, gold, alloy; platinum, titanium, and stainless steel.

According to some aspects of the present disclosure, a method for inserting a member for intraocular insertion, comprises the steps of selecting a member for intraocular insertion to be inserted into a user's cornea, wherein the member for intraocular insertion comprises a ring assembly comprises a plurality of rings having the same center, wherein the plurality of rings have the shape of ellipses with the same eccentricity and different sizes, and a plurality of connection members for connecting between the plurality of rings, incising a portion of the cornea to create an incised portion, and inserting the selected member for intraocular insertion into the incised portion.

According to some aspects, the step of selecting a member for intraocular insertion to be inserted into a user's cornea comprises identifying the corneal state comprising a curvature of the incised portion into which the member for intraocular insertion is inserted, and the step of selecting a member for intraocular insertion corresponding to the identified corneal state from a second set of members for intraocular insertion.

According to some aspects, each member for intraocular insertion of the second set of members for intraocular insertion is different from each other in at least one of the curvature of each member for intraocular insertion, the eccentricity of the plurality of rings included in each member of intraocular insertion, the minimum distance from the center to the smallest ring among the plurality of rings, the minimum distance from the center to the largest ring among the plurality of rings, the intervals between the plurality of rings, whether the intervals between the plurality of rings are constant or not, and whether the auxiliary connection members are disposed where the connection members are not disposed or not.

According to some aspects, the step of incising a portion of the cornea to create an incised portion comprises the step of creating a groove having the same shape as that of the selected member for intraocular insertion in at least one site of corneal epithelium and Bowman's membrane among constitutive tissues of the cornea, or the step of creating a groove having a toric shape with a predetermined size in the at least one site.

According to some aspects, the step of incising a portion of the cornea to create an incised portion comprises the step of incising the corneal stroma among constitutive tissues of the cornea in a horizontal straight line to form an upper corneal stroma and a lower corneal stroma.

According to some aspects, the step of inserting the selected member for intraocular insertion into the incised portion comprises the step of inserting the member for intraocular insertion between the upper corneal stroma and the lower corneal stroma.

According to some aspects, the step of incising the corneal stroma in a horizontal straight line comprises the step of creating a groove having the same shape as that of the selected member for intraocular insertion in the lower corneal stroma, or the step of creating a groove having a toric shape with a predetermined size in the lower corneal stroma.

According to some aspects, tdhe method for inserting a member for intraocular insertion according to claim 22, further comprises the step of suturing between the upper corneal stroma and the lower corneal stroma after the selected member for intraocular insertion is inserted into the incised portion.

According to some aspects, each of the plurality of connection members is disposed apart from an adjacent connection member by the same angle with respect to the center.

According to some aspects, the member for intraocular insertion further comprises an auxiliary connection member for connecting between the plurality of rings and disposed where the connection members are not disposed.

According to some aspects, the material of the ring is at least one of polymethylmethacrylate, gold, alloy, platinum, titanium, and stainless steel.

Aspects of the present disclosure are not limited to those mentioned above, and other objects and advantages of the present disclosure that have not been mentioned can be understood by the following description, and will be more clearly understood by embodiments of the present disclosure. In addition, it will be readily understood that the objects and advantages of the present disclosure can be realized by the means and combinations thereof set forth in the claims.

By inserting a member for intraocular insertion into the eye, corneal ectasia due to intraocular pressure may be prevented.

The specific effects of the present invention in addition to the above-mentioned content will be described together while explaining the specific details for carrying out the invention below.

### BRIEF DESCRIPTION OF THE DRAWINGS

FIGS. 1A to ID are schematic diagrams of constitutive tissues of the cornea and drawings for explaining the process of corneal ectasia due to intraocular pressure.

FIG. 2 shows the member for intraocular insertion according to some embodiments inserted into the eye.

FIGS. 3A to 3C are drawings for explaining the member for intraocular insertion and the ring assembly according to some embodiments.

FIG. 4 shows the member for intraocular insertion according to some embodiments.

FIG. 5 shows the member for intraocular insertion comprising the auxiliary connection member according to some embodiments.

FIGS. 6A to 6E show sets of the members for intraocular insertion according to some embodiments.

FIGS. 7A to 7F show sets of the members for intraocular insertion according to some other embodiments.

FIG. 8 is a flowchart showing the method for inserting a member for intraocular insertion into the eye according to some embodiments.

FIG. 9 is a flowchart showing the method for inserting a member for intraocular insertion into the eye according to some embodiments.

FIGS. 10A to 10D are flowcharts showing the method for inserting a member for intraocular insertion into the eye according to some embodiments and schematic diagrams for explaining the same.

FIGS. 11A and 11B are a flowchart showing the method for inserting a member for intraocular insertion into the eye according to some embodiments and a schematic diagram for explaining the same.

FIGS. 12A and 12B are flowcharts showing the method for inserting a member for intraocular insertion into the eye according to some embodiments.

FIG. 13 is a flowchart showing the method for inserting a member for intraocular insertion into the eye according to some embodiments.

### DETAILED DESCRIPTION OF THE PREFERRED EMBODIMENTS

The terms or words used in the present disclosure and the claims should not be construed as limited to their ordinary or lexical meanings. They should be construed as the meaning and concept in line with the technical idea of the present disclosure based on the principle that the inventor can define the concept of terms or words in order to describe his/her own embodiments in the best possible way. Further, since the embodiment described herein and the configurations illustrated in the drawings are merely one embodiment in which the present disclosure is realized and do not represent all the technical ideas of the present disclosure, it should be understood that there may be various equivalents, variations, and applicable examples that can replace them at the time of filing this application.

Although terms such as first, second, A, B, etc. used in the present description and the claims may be used to describe various components, the components should not be limited by these terms. These terms are used only for the purpose of distinguishing one component from another. For example, a first component may be referred to as a second component, and similarly, a second component may be referred to as a first component, without departing from the scope of the present disclosure. The term ‘and/or’ includes a combination of a plurality of related listed items or any item of the plurality of related listed items.

The terms used in the present description and the claims are merely used to describe particular embodiments and are not intended to limit the present disclosure. Singular expressions include plural expressions unless the context explicitly indicates otherwise. In the present application, terms such as “comprise”, “have”, “include”, “contain”, etc. should be understood as not precluding the possibility of existence or addition of features, numbers, steps, operations, components, parts, or combinations thereof described herein.

When a part is said to include “at least one of a, b or c”, this means that the part may include only a, only b, only c, both a and b, both a and c, both b and c, all of a, b and c, or variations thereof.

Unless otherwise defined, all terms used herein, including technical or scientific terms, have the same meaning as commonly understood by those of ordinary skill in the art to which the present disclosure pertains.

Terms such as those defined in commonly used dictionaries should be construed as having a meaning consistent with the meaning in the context of the relevant art, and are not to be construed in an ideal or excessively formal sense unless explicitly defined in the present disclosure.

In addition, each configuration, procedure, process, method, or the like included in each embodiment of the present disclosure may be shared to the extent that they are not technically contradictory to each other.

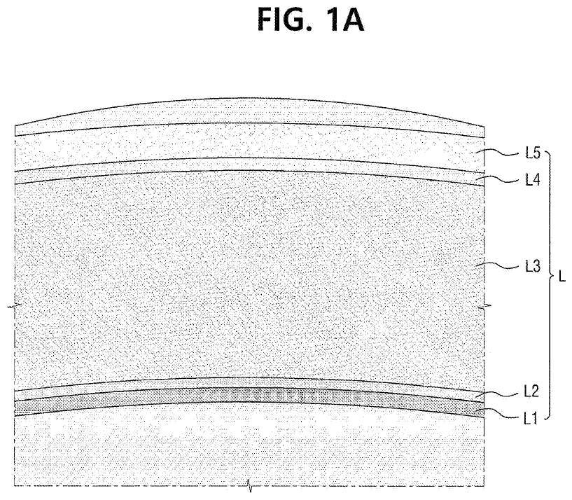

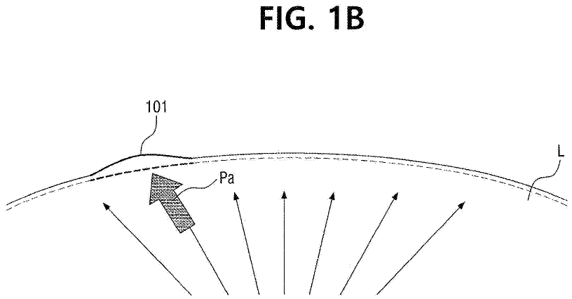

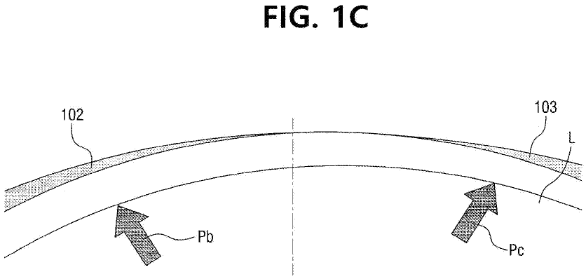

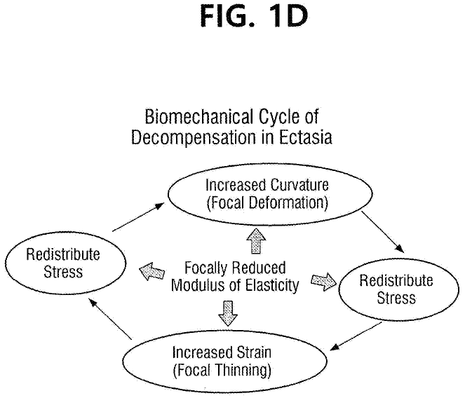

Hereinafter, the members for intraocular insertion and the methods for inserting the member for intraocular insertion into the eye according to some embodiments of the present invention will be described with reference to FIGS. 1A to 13.

FIGS. 1A to ID are schematic diagrams of constitutive tissues of the cornea and drawings for explaining the process of corneal ectasia due to intraocular pressure.

FIG. 1A shows a schematic diagram of constitutive tissues of the cornea. Referring to FIG. 1A, the cornea L of the eye may be divided into five layers. In this case, L1 may represent the corneal endothelium, L2 may represent the posterior border plate (descemet's membrane), L3 may represent the corneal stroma, L4 may represent Bowman's membrane, and L5 may represent the corneal epithelium. However, the cornea of the eye is not limited thereto, and may be divided into more subdivided layers, and some layers may be omitted and called while being merged with other layers.

In general, the thickness of the corneal epithelium L5 is about 50 microns, and the thickness of the corneal stroma L3 is 300 microns or more and 800 microns or less. In this case, if the thickness of the entire cornea L is 360-400 microns or less, it may be seen that the probability of showing keratectasia is high. However, if the intraocular pressure is high, keratectasia may also occur even if the cornea is thick.

FIGS. 1B to ID are drawings for explaining the process of corneal ectasia due to intraocular pressure.

A thin portion of the cornea of the eye may be dilated by intraocular pressure. For example, FIG. 1B shows a portion 101 in which the cornea L is dilated in the direction of the arrow Pa due to intraocular pressure, and FIG. 1C shows portions 102 and 103 in which the cornea L is dilated in the directions of arrows Pb and Pc due to intraocular pressure.

In this case, the dilated portion may create a curvature deviation, which causes abnormal vision and also causes a tear film defect, thereby resulting in xerophthalmia. If the corneal ectasia due to intraocular pressure continues, eventually, the cornea may also be perforated.

FIG. 1D shows the biomechanical cycle of decompensation in corneal ectasia. The cycle shown in FIG. 1D means that if it is initiated by the asymmetry of the distribution of biomechanical properties, then the cornea becomes thinner and the stress increases, and eventually the cornea deforms or redistributes its curvature in a compensatory manner.

Therefore, it is necessary to insert a biocompatible material into the cornea to prevent deformation or ectasia of the cornea due to intraocular pressure, and hereinafter, a member for intraocular insertion that performs such a function will be described in detail.

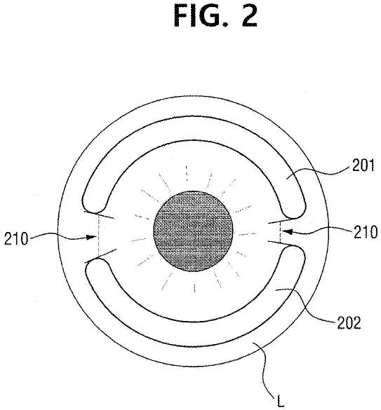

FIG. 2 shows the member for intraocular insertion according to some embodiments inserted into the eye.

Referring to FIG. 2, FIG. 2 shows the upper region 201, the lower region 202, and the empty space 210 between the upper and lower regions, of the member for intraocular insertion inserted into the cornea L of the eye. In this case, each of the upper region 201 and the lower region 202 of the member for intraocular insertion may have a semicircular shape.

In general, when this member for intraocular insertion is inserted into the eye, a tunnel is made in the corneal stroma portion (L3 in FIG. 1A), and then the upper region 201 and the lower region 202 of the member for intraocular insertion are inserted axially symmetrically. In this case, by using the principle that the central cornea is flattened by generating an arc-shortening effect of the cornea, the progression of keratoconus may be suppressed.

However, this member for intraocular insertion may have side effects in that the cornea at the site into which the ring piece is inserted is protruded, and due to this, irregular refractive power and tear film defects are induced. In addition, since the upper region 201 and the lower region 202 of the member for intraocular insertion are separated and inserted axially symmetrically, the curvature of the portions 201 and 202 in which each region is disposed and the curvature of the portion 210 in which each region is not disposed may be different. Therefore, in this case, it may be difficult to evenly improve the shape of the cornea to have a spherical or elliptical curvature.

Therefore, the shape of a new member for intraocular insertion that can solve these problems will be described below.

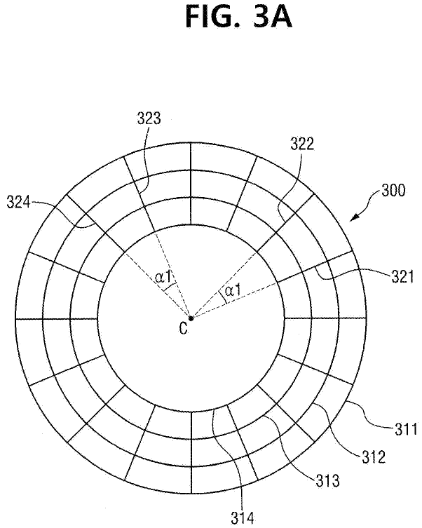

FIGS. 3A to 3C are drawings for explaining the member for intraocular insertion and the ring assembly according to some embodiments.

FIG. 3A shows the member for intraocular insertion 300 according to some embodiments, and FIG. 3B shows a ring assembly 310 of the member for intraocular insertion 300 of FIG. 3A.

Referring to FIGS. 3A and 3B, the member for intraocular insertion 300 may comprise a ring assembly 310 and a plurality of connection members (e.g., 321 to 324).

The ring assembly 310 may comprise a plurality of rings 311 to 314.

In this case, the plurality of rings may have a common center C. In this case, the plurality of rings 311 to 314 may have the shape of concentric circles with different sizes. However, the rings are not limited thereto, and the center of each ring may be partially different.

FIGS. 3A and 3B show that the intervals between the rings 311 to 314 is the same, but are not limited thereto, and the intervals between the rings may be partially different.

A plurality of connection members (e.g., 321 to 324) may connect between the plurality of rings 311 to 314. According to some embodiments, each of the plurality of connection members may be disposed apart from the adjacent connection members by the same angle α1 with respect to the center C, but is not limited thereto.

Each connection member, e.g., 321, may be integrally formed to have a length from the smallest ring 314 to the largest ring 311, or may be formed as a set of detailed connection members having a length equal to the distance between each ring (between 311 and 312, between 312 and 313, and between 313 and 314).

Although FIG. 3A shows that the number of connection members is sixteen, which is only an exemplary value, and the number of connection members may be freely modified.

FIG. 3A shows that each connection member is in a straight line form connecting from the smallest ring 314 to the largest ring 311, but is not limited thereto.

As some embodiments, the connection member may also be in a curved form connecting between each ring (between 311 and 312, between 312 and 313, and between 313 and 314).

As some other embodiments, the connection member may be formed differently between each ring (between 311 and 312, between 312 and 313, and between 313 and 314). For example, the connection member may also be formed to include one straight line form between the ring 311 and the ring 312, two straight line forms between the ring 312 and the ring 313, and one straight line form between the ring 313 and the ring 314.

In some other embodiments, the connection member may also be formed in a zigzag manner to connect between each ring (between 311 and 312, between 312 and 313, and between 313 and 314).

However, the connection member is not limited to the above-described embodiments, and may connect between each ring in various manners.

The material of the plurality of rings and/or the plurality of connection members may be a biocompatible material. In this case, the biocompatible material may be a product made of or coated with a material harmless to the human body. In some embodiments, the biocompatible material may include, for example, polymethylmethacrylate (PMMA), gold, alloy, platinum, titanium, stainless steel, or the like, but is not limited thereto.

The thickness of the member for intraocular insertion 300 may be, for example, 100 microns or less, but is not limited thereto.

FIG. 3C is a schematic diagram for explaining that a plurality of rings 311 to 314 in the ring assembly 310 and connection members (e.g., 321 to 324) are combined to form a kind of mesh shape.

By exhibiting a form in which the plurality of rings and the plurality of connection members in the member for intraocular insertion closely intersect each other, that is, a kind of mesh shape, the member for intraocular insertion may serve as a skeleton that encloses and keeps the shape of the cornea. Therefore, it may be useful in preventing corneal deformation due to intraocular pressure, and improving and keeping the curvature of the cornea to sphericity or ellipsoid.

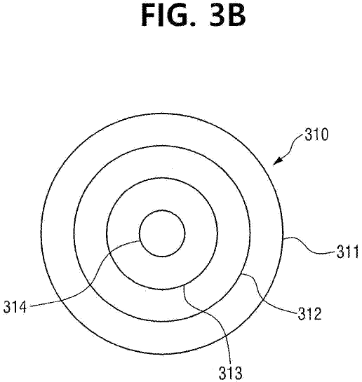

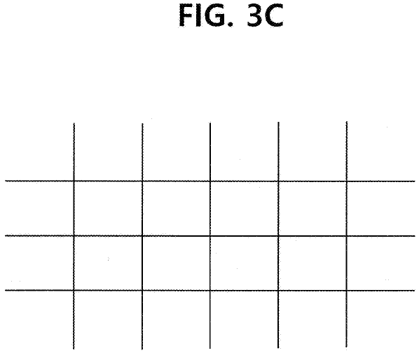

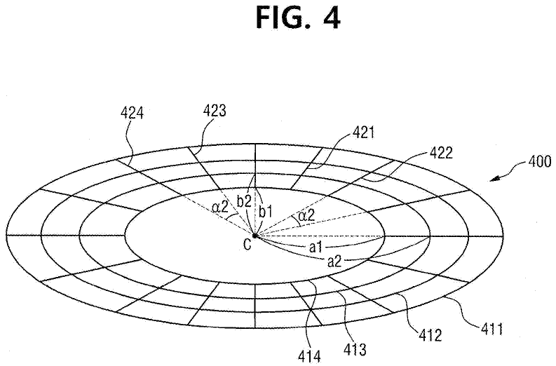

FIG. 4 shows the member for intraocular insertion according to some embodiments.

Referring to FIG. 4, the member for intraocular insertion 400 may comprise a ring assembly including a plurality of rings 411 to 414 and a plurality of connection members (e.g., 421 to 424).

The plurality of rings 411 to 414 may have a common center (C). However, the rings are not limited thereto, and the center of each ring may be partially different.

Each of the plurality of rings 411 to 414 may have an elliptical shape, unlike the plurality of rings 311 to 314 in the form of concentric circles as shown in FIGS. 3A and 3B.

In this case, the plurality of rings 411 to 414 may have the shape of ellipses with the same eccentricity and different sizes.

For example, FIG. 4 shows that in the smallest ring 414, the longest radius represents a1 and the shortest radius represents b1, and shows that in the second smallest ring 413, the longest radius represents a2 and the shortest radius represents b2. In this case, in each of the rings 413 and 414, the longest radii a1 and a2 are different from each other and the shortest radii b1 and b2 are also different from each other, but the eccentricities of each of the rings 413 and 414 calculated based on the ratio of the longest radius to the shortest radius may be equal to each other. However, the rings are not limited thereto, and the eccentricities of each ring may be partially different from each other.

FIG. 4 shows that the intervals between the rings 411 to 414 are the same, but are not limited thereto, and the intervals between the rings may be partially different.

A plurality of connection members (e.g., 421 to 424) may connect between the plurality of rings 411 to 414. Each of the plurality of connection members may be disposed apart from the adjacent connection members by the same angle α2 with respect to the center C, but is not limited thereto.

In this case, each connection member may be integrally formed to have a length from the smallest ring 414 to the largest ring 411, or may be formed as a set of detailed connection members having a length equal to the distance between each ring (between 411 and 412, between 412 and 413, and between 413 and 414).

Although FIG. 4 shows that the number of connection members is sixteen, which is only an exemplary value, and the number of connection members may be freely modified.

FIG. 4 shows that each connection member is in a straight line form connecting from the smallest ring 414 to the largest ring 411, but is not limited thereto.

As an example, the connection member may also be in a curved form connecting between each ring (between 411 and 412, between 412 and 413, and between 413 and 414).

As another example, the connection member may be formed differently between each ring (between 411 and 412, between 412 and 413, and between 413 and 414). For example, the connection member may also be formed to include one straight line form between the ring 411 and the ring 412, two straight line forms between the ring 412 and the ring 413, and one straight line form between the ring 413 and the ring 414.

As another example, the connection member may be formed in a zigzag manner between each ring (between 411 and 412, between 412 and 413, and between 413 and 414).

However, the connection member is not limited to the above-described embodiments, and may connect between each ring in various manners.

The material of the plurality of rings and/or the plurality of connection members may be a biocompatible material. In this case, the biocompatible material may be a product made of or coated with a material harmless to the human body. In some embodiments, the biocompatible material may include, for example, polymethylmethacrylate (PMMA), gold, alloy, platinum, titanium, stainless steel, or the like, but is not limited thereto.

The thickness of the member for intraocular insertion 400 may be, for example, 100 microns or less, but is not limited thereto.

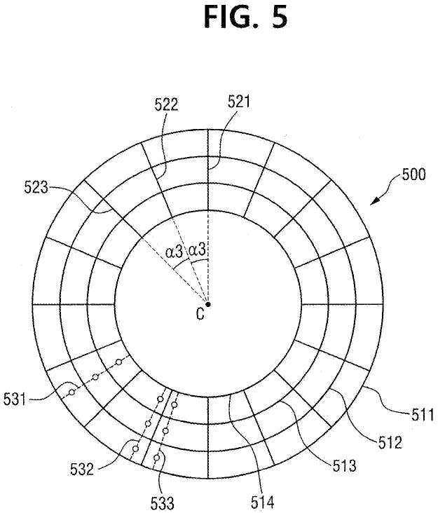

FIG. 5 shows the member for intraocular insertion comprising the auxiliary connection member according to some embodiments.

Referring to FIG. 5, the member for intraocular insertion 500 may comprise a ring assembly including a plurality of rings 511 to 514, a plurality of connection members 521 to 523, and auxiliary connection members 531 to 533.

FIG. 5 shows that the plurality of rings 511 to 514 are in the form of concentric circles as shown in FIG. 3A, but the rings are not limited thereto, and each of the rings 511 to 514 in FIG. 5 may also have the shape of ellipses with the same eccentricity and different sizes, as described in FIG. 4 above.

FIG. 5 shows that each of the plurality of connection members (e.g., 521 to 523) is disposed apart from the adjacent connection members by the same angle α3 with respect to the center C. Although FIG. 5 shows that the number of connection members is sixteen, it is of course not limited thereto. The illustrated angle α3 may be the same as or different from the angle α1 in FIG. 3A and/or the angle α2 in FIG. 4.

The auxiliary connection members 531 to 533 may be disposed where each of the connection members (e.g., 521 to 523) is not disposed.

FIG. 5 showed auxiliary connection members 531 to 533 with dotted lines for convenience of explanation. Although FIG. 5 shows that the number of auxiliary connection members 531 to 533 is three, which is only an exemplary value and may be freely modified.

Through the disposition of the auxiliary connection member 531, it is possible to cover a case where the thickness of a user's cornea is not uneven. For example, when the thickness of the eye cornea is thin in a first portion and thick in a second portion, the member for intraocular insertion 500 may be inserted such that the first portion corresponds to a position where the auxiliary connection members 531 to 533 are disposed, and the second portion corresponds to a position where the auxiliary connection members 531 to 533 are not disposed.

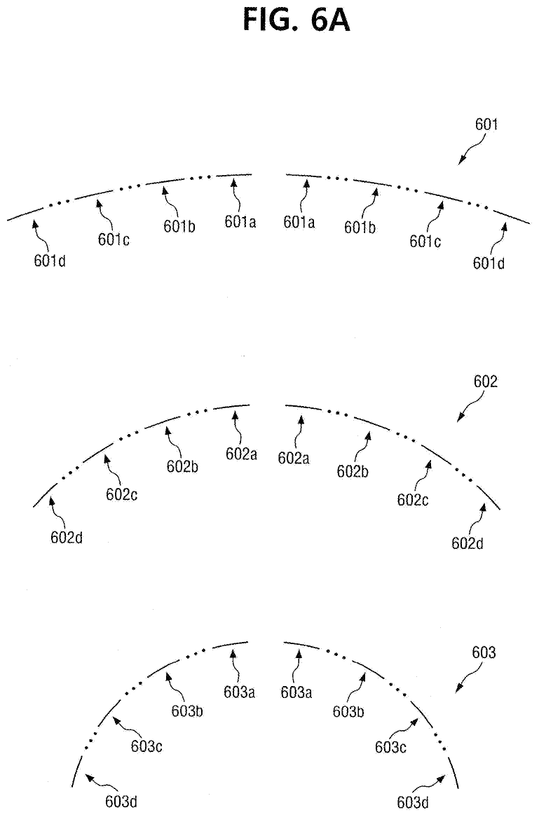

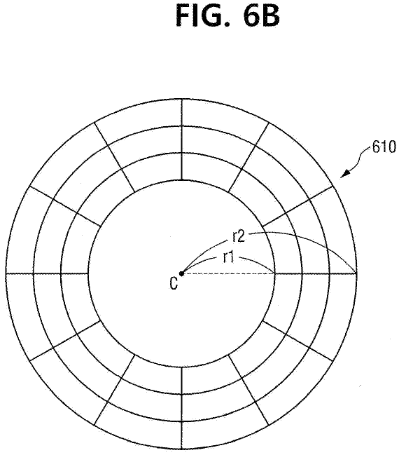

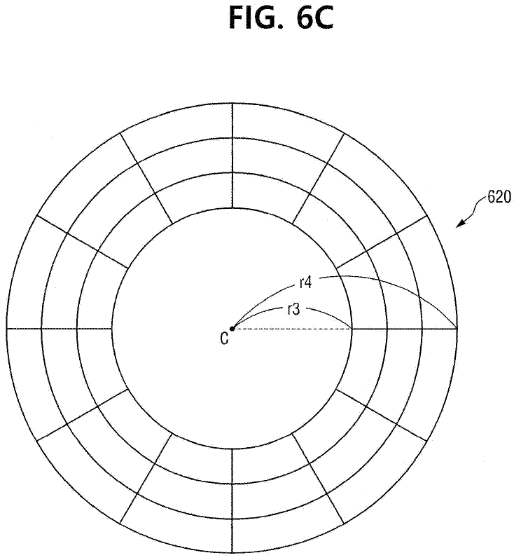

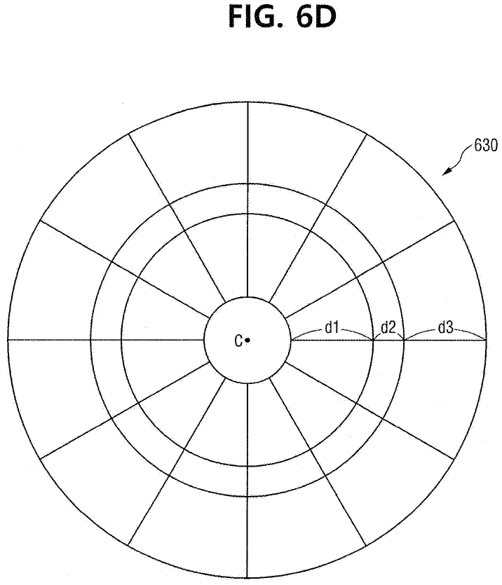

FIGS. 6A to 6E show sets of the members for intraocular insertion according to some embodiments. FIGS. 6A to 6E show a first set of the members for intraocular insertion 601, 602, 603, 610, 620, 630, 640 in which the shape of a plurality of rings is circular.

FIG. 6A shows the front view of the members for intraocular insertion 601, 602, 603 having different curvatures.

Each of the members for intraocular insertion 601 to 603 is shown to include four rings. That is, the member for intraocular insertion 601 is shown to include four rings 601a, 601b, 601c, and 601d, the member for intraocular insertion 602 is shown four rings 602a, 602b, 602c, and 602d, and the member for intraocular insertion 603 is shown to include four rings 603a, 603b, 603c, and 603d, wherein the connection member between each ring is shown with dotted lines for convenience.

Referring to FIG. 6A, the curvature of the member for intraocular insertion 602 is greater than that of the member for intraocular insertion 601 and smaller than that of the member for intraocular insertion 603.

That is, the first set of the members for intraocular insertion may include a plurality of the members for intraocular insertion 601 to 603 having different curvatures. According to some embodiments, the first set of the members for intraocular insertion may include a plurality of the members for intraocular insertion having a curvature difference of 0.25D units.

Referring to FIGS. 6B and 6C, FIG. 6B shows the distance r1 from the center C to the smallest ring, and the distance r2 from the center C to the largest ring in the member for intraocular insertion 610; and FIG. 6C shows the distance r3 from the center C to the smallest ring, and the distance r4 from the center C to the largest ring in the member for intraocular insertion 620.

In this case, r3 may be greater than r1, and r4 may be greater than r2.

That is, the first set of the members for intraocular insertion may include a plurality of the members for intraocular insertion 610 and 620 in which the distances from the center to the smallest ring among the plurality of rings and the distances from the center to the largest ring among the plurality of rings are different.

Referring to FIG. 6D, FIG. 6D shows that the intervals between each ring in the member for intraocular insertion 630 represent d1, d2, and d3. As shown in FIG. 6D, d2 may be smaller than d1 or d3.

That is, the first set of the members for intraocular insertion may include the member for intraocular insertion 630 in which the intervals between the plurality of rings are not constant.

However, it is natural that the first set of the members for intraocular insertion not only includes the member for intraocular insertion 630 in which the intervals d1, d2, and d3 between each ring in one member for intraocular insertion 630 are not constant as shown in FIG. 6D, but also may include a plurality of members for intraocular insertion with a constant interval between each ring but different intervals from each other.

Referring to FIG. 6E, FIG. 6E shows the member for intraocular insertion 640 comprising a plurality of connection members disposed apart from each other by the same angle with respect to the center C, and auxiliary connection members 641 to 645 disposed where the plurality of connection members are not disposed.

That is, the first set of the members for intraocular insertion may include the member for intraocular insertion 640 comprising the auxiliary connection members disposed where the plurality of connection members are not disposed.

Taken together FIGS. 6A to 6E, the first set of the members for intraocular insertion 601, 602, 603, 610, 620, 630, and 640 may include a plurality of members for intraocular insertion which are different from each other in at least one of the curvature of each member for intraocular insertion, the distance from the center to the smallest ring among the plurality of rings, the distance from the center to the largest ring among the plurality of rings, the intervals between the plurality of rings, whether the intervals between the plurality of rings are constant or not, and whether auxiliary connection members are disposed where the connection members are not disposed or not.

However, the first set of members for intraocular insertion is not limited to the above-described embodiments, and may include members for intraocular insertion having more various forms and shapes.

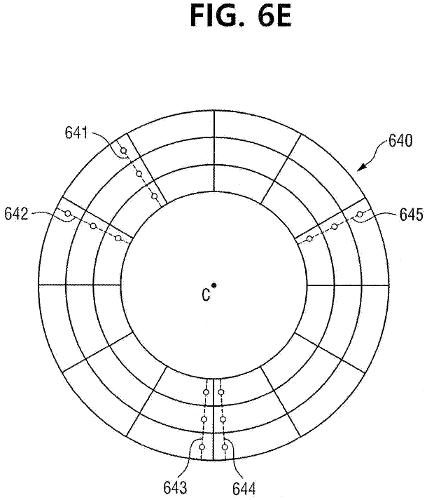

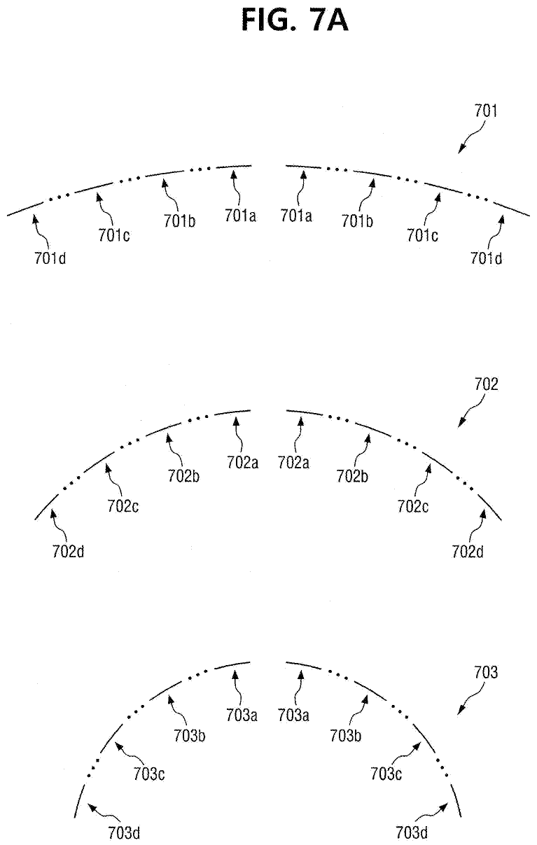

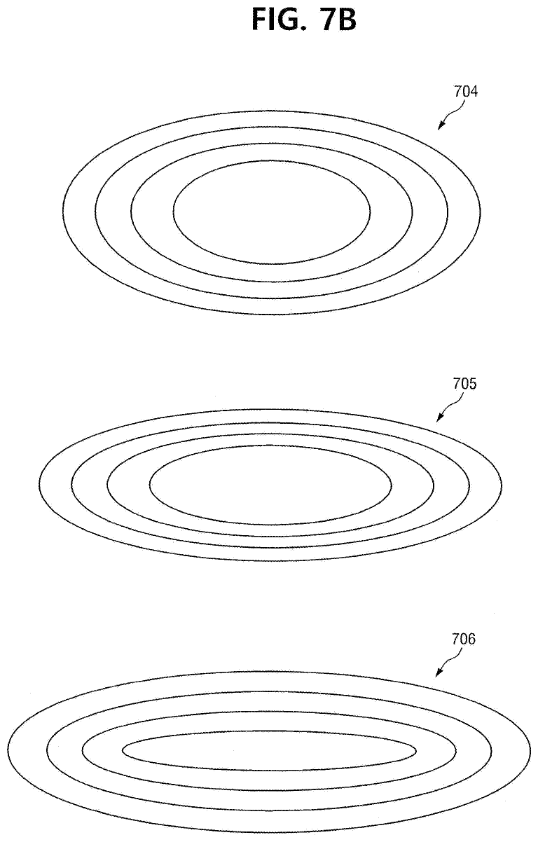

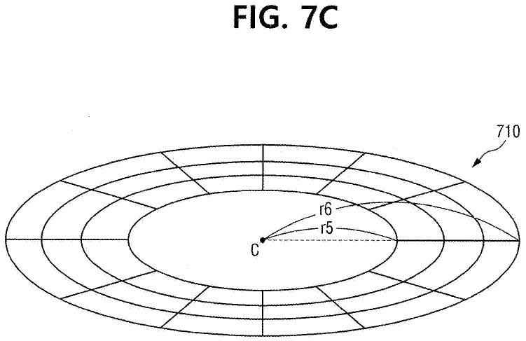

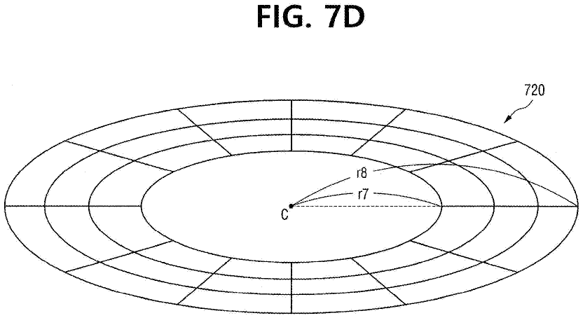

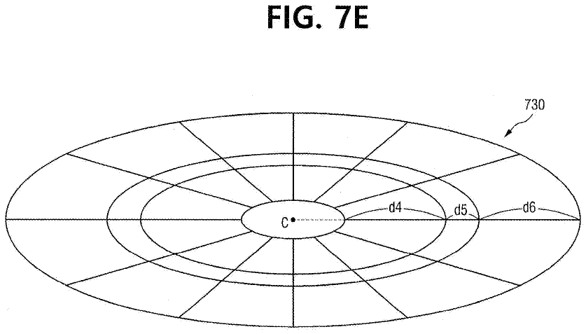

FIGS. 7A to 7F show sets of the members for intraocular insertion according to some other embodiments. FIGS. 7A to 7F show a second set of the members for intraocular insertion 701, 702, 703, 704, 705, 706, 710, 720, 730, 740 in which the shape of a plurality of rings is elliptic.

FIG. 7A shows the front view of the members for intraocular insertion 701, 702, 703 having different curvatures.

Each of the members for intraocular insertion 701 to 703 is shown to include four rings. That is, the member for intraocular insertion 701 is shown to include four rings 701a, 701b, 701c, and 701d, the member for intraocular insertion 702 is shown four rings 702a, 702b, 702c, and 702d, and the member for intraocular insertion 703 is shown to include four rings 703a, 703b, 703c, and 703d, wherein the connection member between each ring is shown with dotted lines for convenience.

Referring to FIG. 7A, the curvature of the member for intraocular insertion 702 is greater than that of the member for intraocular insertion 701 and smaller than that of the member for intraocular insertion 703.

That is, the second set of the members for intraocular insertion may include a plurality of the members for intraocular insertion 701 to 703 having different curvatures. According to some embodiments, the second set of the members for intraocular insertion may include a plurality of the members for intraocular insertion having a curvature difference of 0.25D units.

FIG. 7B shows a schematic diagram of the members for intraocular insertion 704, 705, and 706 with different eccentricities.

Referring to FIG. 7A, the eccentricities of the rings included in each of the members for intraocular insertion 704, 705, and 706 are constant, but the eccentricity of each ring in the member for intraocular insertion 705 is greater than the eccentricity of each ring in the member for intraocular insertion 704 and smaller than the eccentricity of each ring in the member for intraocular insertion 706.

That is, the second set of the members for intraocular insertion may include a plurality of the members for intraocular insertion 704 to 706 having different eccentricities of each ring.

Referring to FIGS. 7C and 7D, FIG. 7C shows the distance r5 from the center C to the smallest ring, and the distance r6 from the center C to the largest ring in the member for intraocular insertion 710; and FIG. 7D shows the distance r7 from the center C to the smallest ring, and the distance r8 from the center C to the largest ring in the member for intraocular insertion 720.

In this case, r7 may be greater than r5, and r8 may be greater than r6.

That is, the second set of the members for intraocular insertion may include a plurality of the members for intraocular insertion 710 and 720 in which the distances from the center to the smallest ring among the plurality of rings and the distances from the center to the largest ring among the plurality of rings are different.

Referring to FIG. 7E, FIG. 7E shows that the intervals between each ring in the member for intraocular insertion 730 represent d4, d5, and d6. As shown in FIG. 7E, d5 may be smaller than d4 or d6.

That is, the second set of the members for intraocular insertion may include the member for intraocular insertion 730 in which the intervals between the plurality of rings are not uniform.

However, it is natural that the first set of the members for intraocular insertion not only includes the member for intraocular insertion 730 in which the intervals d4, d5, and d6 between each ring in one member for intraocular insertion 730 are not constant as shown in FIG. 7E, but also may include a plurality of members for intraocular insertion with a constant interval between each ring but different intervals from each other.

Referring to FIG. 7F, FIG. 7F shows the member for intraocular insertion 740 comprising a plurality of connection members disposed apart from each other by the same angle with respect to the center C, and auxiliary connection members 741 to 745 disposed where the plurality of connection members are not disposed.

That is, the second set of the members for intraocular insertion may include the member for intraocular insertion 740 comprising the auxiliary connection members disposed where the plurality of connection members are not disposed.

Taken together FIGS. 7A to 7F, the second set of the members for intraocular insertion 701, 702, 703, 704, 705, 706, 710, 720, 730, and 740 may include a plurality of members for intraocular insertion which are different from each other in at least one of the curvature of each member for intraocular insertion, eccentricity of a plurality of rings included in each member for intraocular insertion, the distance from the center to the smallest ring among the plurality of rings, the distance from the center to the largest ring among the plurality of rings, the intervals between the plurality of rings, whether the intervals between the plurality of rings are constant or not, and whether auxiliary connection members are disposed where the connection members are not disposed or not.

However, the second set of members for intraocular insertion is not limited to the above-described embodiments, and may include members for intraocular insertion having more various forms and shapes.

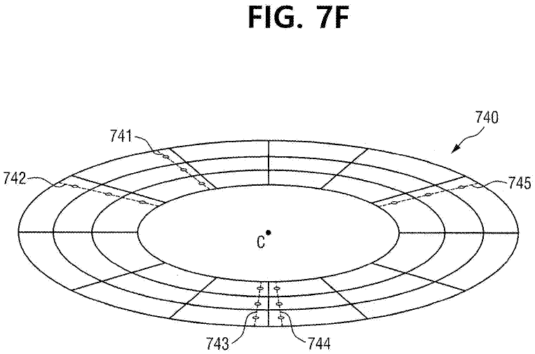

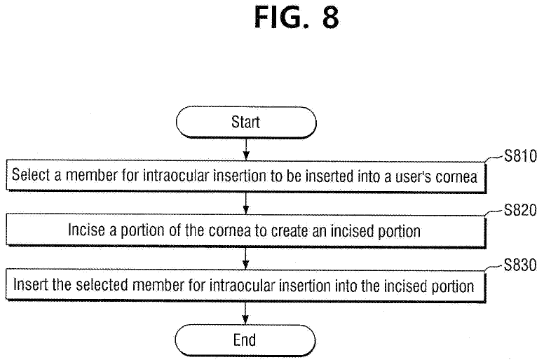

FIG. 8 is a flowchart showing the method for inserting a member for intraocular insertion into the eye according to some embodiments.

First, a member for intraocular insertion to be inserted into a user's cornea may be selected (S810).

In this case, any one member for intraocular insertion may be selected from the members for intraocular insertion comprising a ring assembly including a plurality of rings having the same center and a plurality of connection members for connecting between the plurality of rings, wherein the plurality of rings have the shape of concentric circles with different sizes, and the members for intraocular insertion comprising a ring assembly including a plurality of rings having the same center and a plurality of connection members for connecting between the plurality of rings, wherein the plurality of rings have the shape of ellipses with the same eccentricity and different sizes. However, it is not limited thereto.

Then, a portion of the cornea may be incised to create an incised portion (S820).

In this case, the portion of the cornea may be part or all of corneal epithelium, Bowman's membrane, corneal stroma, and the like, among constitutive tissues of the cornea. The portion of the cornea may be incised through a laser (e.g., femtosecond laser), a microkeratome (e.g., microkeratome), or the like.

Before step S820 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S830).

After the step S830, the step of wearing a contact lens to protect the cornea while the corneal epithelium is restored may be followed.

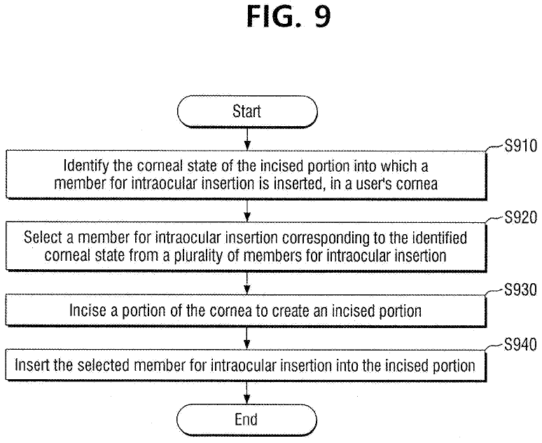

FIG. 9 is a flowchart showing the method for inserting a member for intraocular insertion into the eye according to some embodiments.

First, in a user's cornea, the corneal state of the incised portion into which a member for intraocular insertion is inserted may be identified (S910).

As some embodiments, the corneal state of the incised portion may include a curvature of the incised portion. For example, the corneal state of the incised portion may include, but is not limited to, a mean spherical curvature, a mean curvature of an ellipsoid, and the like.

As some other embodiments, the corneal state of the incised portion may include whether the thickness of the cornea is even or not. For example, the corneal state of the incised portion may include whether a little thin portion and a little thick portion of a user's cornea are distinguished or not, and the like.

As further some other embodiments, the corneal state of the incised portion may include the size and diameter of a user's cornea, and the like.

However, it is natural that the corneal state is not limited to the above-described embodiments and may include more various embodiments.

In this case, the corneal state of the incised portion may be determined manually by a human or may be identified using an eye scanner, a 3D scanner, and other medical imaging devices.

Then, a member for intraocular insertion corresponding to the identified corneal state may be selected from a plurality of members for intraocular insertion (S920).

As some embodiments, the member for intraocular insertion having the highest matching rate between the identified corneal state and the shape, form, and the like may be selected from the plurality of members for intraocular insertion. In this case, the matching rate may be determined manually by a human or may determined through a device utilizing a predetermined program, a medical device, a scanning device, and the like.

The plurality of members for intraocular insertion may include members for intraocular insertion in which the plurality of rings have the shape of concentric circles, and/or members for intraocular insertion in which the plurality of rings have the shape of ellipses with the same center and different sizes.

As some embodiments, each member for intraocular insertion of the members for intraocular insertion may be different from each other in at least one of whether each ring of the member for intraocular insertion is circular or elliptic, the curvature of each member for intraocular insertion, the distance from the center to the smallest ring among the plurality of rings, the distance from the center to the largest ring among the plurality of rings, the intervals between the plurality of rings, whether the intervals between the plurality of rings are constant or not, whether the auxiliary connection members are disposed where the connection members are not disposed or not, and the eccentricity of the rings in each member for intraocular insertion in which the plurality of rings have the shape of ellipses with the same center and different sizes.

For example, the plurality of ocular insertion members may include the first set of members for intraocular insertion in above-described FIGS. 6A-6E, the second set of members for intraocular insertion of above-described FIGS. 7A-7F, and the like, but are limited thereto, and the plurality of members for intraocular insertion may include members for intraocular insertion of various embodiments.

That is, by selecting the member for intraocular insertions corresponding to the corneal state of the identified incised portion (e.g., curvature, whether the corneal thickness is even or not, the size of the cornea, and the like) from the plurality of members for intraocular insertion described above, for example, the members for intraocular insertion that are different from each other in curvature, size, and presence or absence of an auxiliary connection member, it is possible to insert a member for intraocular insertion optimized for a user's corneal state.

In this case, by inserting the member for intraocular insertion optimized for a user's corneal state, it has the remarkable effects that it possible to more firmly prevent corneal deformation due to intraocular pressure, thereby enclosing and keeping the shape of the cornea, and it may also useful in more effectively improving and keeping the curvature of the cornea to sphericity or ellipsoid.

Then, a portion of the cornea may be incised to create an incised portion (S930).

In this case, the portion of the cornea may be part or all of corneal epithelium, Bowman's membrane, corneal stroma, and the like, among constitutive tissues of the cornea. The portion of the cornea may be incised through a laser (e.g., femtosecond laser), a microkeratome (e.g., microkeratome), or the like.

Before step S930 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S940).

After the step S940, the step of wearing a contact lens to protect the cornea while the corneal epithelium is restored may be followed.

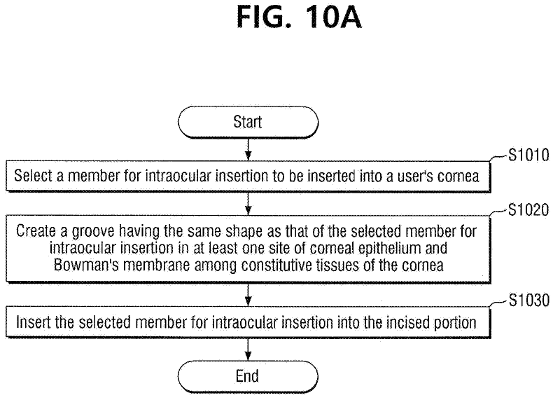

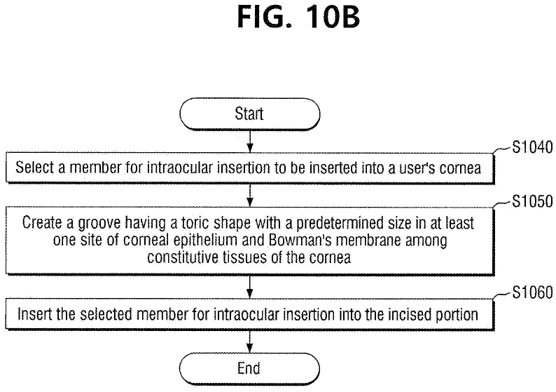

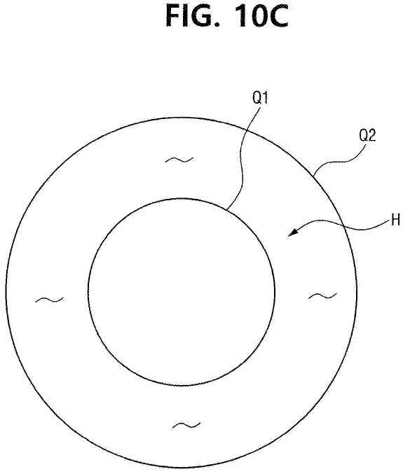

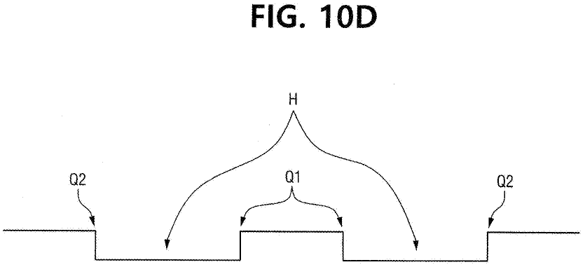

FIGS. 10A to 10D are flowcharts showing the method for inserting a member for intraocular insertion into the eye according to some embodiments and schematic diagrams for explaining the same.

Referring to FIG. 10A, first, a member for intraocular insertion to be inserted into a user's cornea may be selected (S1010). In this case, since step S1010 may refer to the same steps as step S810 in FIG. 8 and/or steps S910 and S920 in FIG. 9, a detailed description thereof will be omitted.

Then, a groove having the same shape as that of the selected member for intraocular insertion may be created in at least one site of corneal epithelium and Bowman's membrane among constitutive tissues of the cornea (S1020). In this case, the groove in at least one site of the corneal epithelium and Bowman's membrane may be created through a laser or the like.

That is, by creating a groove to have a shape matching the shape (e.g., curvature, size, presence or absence of an auxiliary connection member, and the like) of the selected member for intraocular insertion and inserting it to fit the groove, corneal removal may be minimized. In this case, since the shape of the created groove and the shape of the member for intraocular insertion to be inserted are the same, the side effect of protruding the member for intraocular insertion after insertion may be minimized.

Before step S1020 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S1030).

In this case, since the shape of the created groove and the member for intraocular insertion as described above is the same, it may be inserted by inserting the member for intraocular insertion into the created groove.

After the step S1030, the step of wearing a contact lens to protect the cornea while the corneal epithelium is restored may be followed.

Referring to FIG. 10B, first, a member for intraocular insertion to be inserted into a user's cornea may be selected (S1040). In this case, since step S1040 may refer to the same steps as step S810 in FIG. 8 and/or steps S910 and S920 in FIG. 9, a detailed description thereof will be omitted.

Then, a groove having a toric shape with a predetermined size may be created in at least one site of corneal epithelium and Bowman's membrane among constitutive tissues of the cornea (S1050).

In this case, the toric shape may include a general toric shape, an ellipsoidal toric shape, and the like. In this case, the groove in at least one site of the corneal epithelium and Bowman's membrane may be created through a laser or the like.

The generated groove having a toric shape may be created to have a size equal to or larger than the size of the selected member for intraocular insertion so that the member for intraocular insertion may be inserted thereinto. Unlike in FIG. 10A, in FIG. 10B, a portion of the cornea may be more conveniently incised by creating a groove having a toric shape.

The generated groove having a toric shape will be described with reference to FIGS. 10C and 10D.

FIG. 10C shows a schematic diagram of the generated groove having a toric shape, and FIG. 10D shows a cross-sectional view of FIG. 10C.

Referring to FIGS. 10C and 10D, the generated groove H having a toric shape and the interfaces Q1 and Q2 of the grooves H are shown. The selected member for intraocular insertion may be inserted into the generated groove H having a toric shape.

In this case, in order that the selected member for intraocular insertion may be inserted into the groove H, the distance between the interfaces Q1 may be made to be larger than the size of the smallest ring of the selected member for intraocular insertion, and the distance between the interfaces Q2 may be made to be larger than the size of the largest ring of the selected member for intraocular insertion.

Referring back to FIG. 10B, before step S1050 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S1060).

In this case, the selected member for intraocular insertion may be inserted into the groove having a toric shape created with a size larger than that of the slected member for intraocular insertion.

After the step S1060, the step of wearing a contact lens to protect the cornea while the corneal epithelium is restored may be followed.

In the case of FIGS. 10A and 10B, the process of creating a groove in the corneal epithelium and Bowman's membrane, not the corneal stroma, is shown.

Thereafter, when the member for intraocular insertion is removed, since the corneal epithelium has a property that is restored to its original state, the process of removing the member for intraocular insertion may be easier than when a groove is created in the corneal stroma.

That is, when the member for intraocular insertion inserted into the corneal stroma is removed, since the corneal stroma has unrestored properties, an additional incision process (e.g., incising the portion except for H to match the height of H in order to match the heights of H and Q1, Q2 in FIG. 10D) may be required to match the height of the incised portion (groove, e.g., H in FIG. 10D) and the non-incised portion (e.g., Q1 and Q2 in FIG. 10D), but when the corneal epithelium is incised, it has the effect that such an additional incision process is not required.

However, unlike those (steps 1020 and 1050) shown in FIGS. 10A to 10D, the grooves having the same shape as those of the member for intraocular insertion and/or the grooves having a toric shape may be formed through the corneal stroma and Bowman's membrane to the corneal stroma.

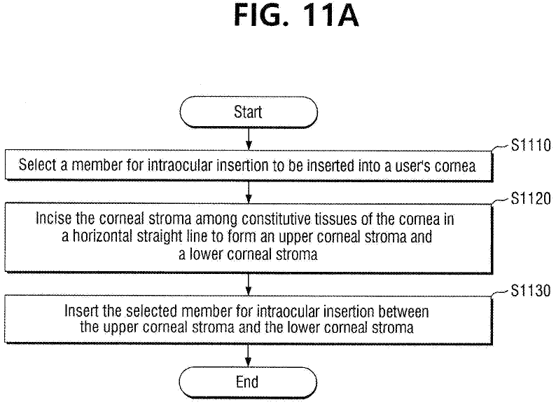

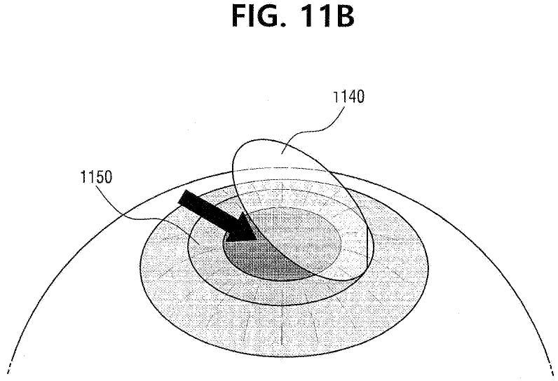

FIGS. 11A and 11B are a flowchart showing the method for inserting a member for intraocular insertion into the eye according to some embodiments and a schematic diagram for explaining the same.

First, a member for intraocular insertion to be inserted into a user's cornea may be selected (S1110).

In this case, since step S1110 may refer to the same steps as step S810 in FIG. 8 and/or steps S910 and S920 in FIG. 9, a detailed description thereof will be omitted.

Then, the corneal stroma among constitutive tissues of the cornea may be incised in a horizontal straight line to form an upper corneal stroma and a lower corneal stroma (S1120). FIG. 11B shows an example of the upper corneal stroma 1140 and the lower corneal stroma 1150 formed by incising the corneal stroma in a horizontal straight line.

In this case, corneal stroma may be incised in a horizontal straight line using a laser (e.g., femtosecond laser), a microkeratome (e.g., microkeratome), or the like.

Before step S1120 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S1130).

In this case, the selected member for intraocular insertion may be inserted between the upper corneal stroma and the lower corneal stroma, for example, at the position of the arrow in FIG. 11B.

After the step S1130, the step of suturing between the upper corneal stroma and the lower corneal stroma, the step of wearing a contact lens to protect the cornea while the corneal epithelium is restored, and the like may be followed.

When the corneal stroma is incised in a horizontal straight line as shown in FIGS. 11A and 11B and then a member for intraocular insertion is inserted therebetween, since it is not necessary to separately create a groove having the same shape as that of the member for intraocular insertion and/or a groove having a toric shape, it may be easier and more convenient to insert the member for intraocular insertion into the eye. In addition, since it is not a process of creating a groove in the corneal stroma, i.e., a process of digging out a portion of the corneal stroma, it has the effect that the corneal stroma having unrestored properties may be minimally removed. Additionally, even when the inserted mesh ring is removed, it is incised in a horizontal straight line, and then the member for intraocular insertion may be extracted, so that its removal process is easy.

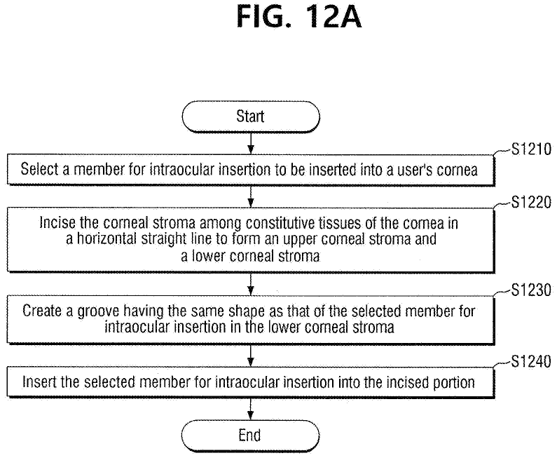

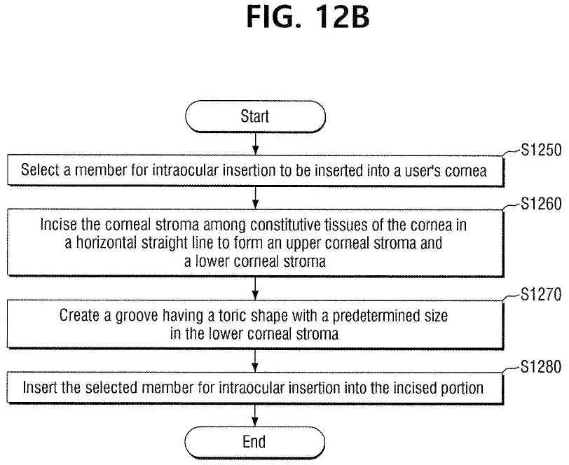

FIGS. 12A and 12B are flowcharts showing the method for inserting a member for intraocular insertion into the eye according to some embodiments.

Referring to FIG. 12A, first, a member for intraocular insertion to be inserted into a user's cornea may be selected (S1210). In this case, since step S1210 may refer to the same steps as step S810 in FIG. 8 and/or steps S910 and S920 in FIG. 9, a detailed description thereof will be omitted.

Then, the corneal stroma among constitutive tissues of the cornea may be incised in a horizontal straight line to form an upper corneal stroma and a lower corneal stroma (S1220). In this case, since step S1220 may refer to the same steps as step S1120 in FIG. 11A, a detailed description thereof will be omitted.

Then, a groove having the same shape as that of the selected member for intraocular insertion may be created in the lower corneal stroma (S1230). In this case, the groove in the lower corneal stroma may be created through a laser or the like.

That is, by creating a groove to have a shape matching the shape (e.g., curvature, size, presence or absence of an auxiliary connection member, and the like) of the selected member for intraocular insertion and inserting it to fit the groove, the side effect of protruding the member for intraocular insertion after insertion may be minimized.

Then, the selected member for intraocular insertion may be inserted into the incised portion (S1240).

In this case, since the shape of the created groove and the member for intraocular insertion as described above is the same, it may be inserted by inserting the member for intraocular insertion into the created groove.

After the step S1240, the step of suturing between the upper corneal stroma and the lower corneal stroma, the step of wearing a contact lens to protect the cornea while the corneal epithelium is restored, and the like may be followed.

Referring to FIG. 12B, first, a member for intraocular insertion to be inserted into a user's cornea may be selected (S1250). Since step S1250 may refer to the same steps as step S810 in FIG. 8 and/or steps S910 and S920 in FIG. 9, a detailed description thereof will be omitted.

Then, the corneal stroma among constitutive tissues of the cornea may be incised in a horizontal straight line to form an upper corneal stroma and a lower corneal stroma (S1260). In this case, since step S1260 may refer to the same steps as step S1120 in FIG. 11A, a detailed description thereof will be omitted.

Then, a groove having a toric shape with a predetermined size may be created in the lower corneal stroma (S1270). In this case, the toric form may include a general toric form, an ellipsoidal toric form, and the like. The groove in the lower corneal stroma may be created through a laser or the like.

The generated groove having a toric shape may be created to have a size equal to or larger than the size of the selected member for intraocular insertion so that the member for intraocular insertion may be inserted thereinto. Unlike in FIG. 12A, in FIG. 12B, a portion of the cornea may be more conveniently incised by creating a groove having a toric shape.

Then, the selected member for intraocular insertion may be inserted into the incised portion (S1280). In this case, the selected member for intraocular insertion may be inserted into the groove having a toric shape created with a size larger than that of the slected member for intraocular insertion.

In the case of FIGS. 12A and 12B, by adding the step of creating a groove (e.g., a groove having the same shape as that of a member for intraocular insertion or a groove having a toric shape with a predetermined size) in the lower corneal stroma among the bisected corneal stroma, it has the effect of reducing the protrusion of the inserted member for intraocular insertion compared to FIGS. 11A and 11B.

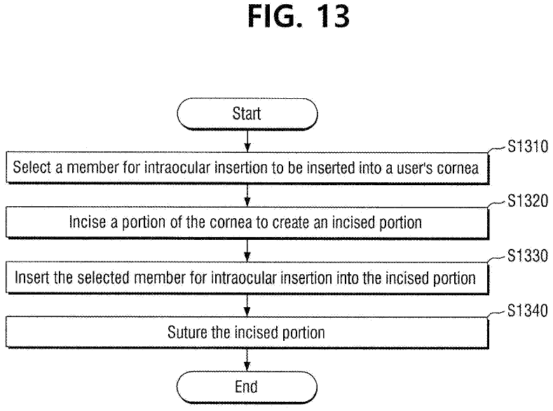

FIG. 13 is a flowchart showing the method for inserting a member for intraocular insertion into the eye according to some embodiments.

First, a member for intraocular insertion to be inserted into a user's cornea may be selected (S1310).

In this case, since step S1310 may refer to the same steps as step S810 in FIG. 8 and/or steps S910 and S920 in FIG. 9, a detailed description thereof will be omitted.

Then, a portion of the cornea may be incised to create an incised portion (S1320).

Then, the step S1320 may include at least one step of the step of incising the corneal stroma among constitutive tissues of the cornea in a horizontal straight line to form an upper corneal stroma and a lower corneal stroma, and the step of creating a groove in the lower corneal stroma. The step of creating a groove in the lower corneal stroma may include creating a groove in the lower corneal stroma having the same shape as that of the selected member for eye insertion, or creating a groove having a toric shape with a predetermined size in the lower corneal stroma.

Step S1320 may be performed through a laser (e.g., femtosecond laser), a microkeratome (e.g., microkeratome), or the like.

Before step S1320 is performed, a pre-processing step such as the step of disinfecting the patient's eyelids and surroundings and/or the step of opening to fix the upper and lower eyelids with speculum may be preceded.

Then, the selected member for intraocular insertion may be inserted into the incised portion (S1330). According to some embodiments, in this case, a member for intraocular insertion may be inserted between the upper corneal stroma and the lower corneal stroma, may be inserted into a groove having the same shape as that of the member for intraocular insertion and formed in the lower corneal stroma, or may be inserted into a groove having a toric shape with a predetermined size and formed in the lower corneal stroma.

Then, the incised portion may be sutured (S1340).

In this case, the upper corneal stroma and the lower corneal stroma may be sutured. According to some embodiments, the upper corneal stroma and the lower corneal stroma may be compressed using a suture or the like.

After the step S1340, the step of wearing a contact lens to protect the cornea while the corneal epithelium is restored may be followed.

The steps of inserting each of the members for intraocular insertion in FIGS. 8 to 13 may be performed by a human and/or may be performed by an automated medical device, a device for intraocular insertion, a surgical robot, or the like.

While the present inventive concept has been particularly shown and described with reference to exemplary embodiments thereof, it will be understood by those of ordinary skill in the art that various changes in form and details may be made therein without departing from the spirit and scope of the present inventive concept as defined by the following claims. It is therefore desired that the present embodiments be considered in all respects as illustrative and not restrictive, reference being made to the appended claims rather than the foregoing description to indicate the scope of the disclosure.

1. A member for intraocular insertion comprising:
a ring assembly enclosing a portion of the cornea and comprising a plurality of rings having the same center, wherein the plurality of rings have the shape of concentric circles with different sizes, and
a plurality of connection members for connecting between the plurality of rings, wherein each of the plurality of connection members is disposed apart from an adjacent connection member by the same angle with respect to the center.

2. The member for intraocular insertion according to claim 1, further comprising an auxiliary connection member for connecting between the plurality of rings and disposed where the connection members are not disposed.

3. The member for intraocular insertion according to claim 1, wherein the material of the ring is at least one of polymethylmethacrylate (PMMA), gold, alloy, platinum, titanium, and stainless steel.

4. A member for intraocular insertion comprising:
a ring assembly enclosing a portion of the cornea and comprising a plurality of rings having the same center, wherein the plurality of rings have the shape of ellipses with the same eccentricity and different sizes, and
a plurality of connection members for connecting between the plurality of rings, wherein each of the plurality of connection members is disposed apart from an adjacent connection member by the same angle with respect to the center.

5. The member for intraocular insertion according to claim 4, further comprising an auxiliary connection member for connecting between the plurality of rings and disposed where the connection members are not disposed.

6. The member for intraocular insertion according to claim 4, wherein the material of the ring is at least one of polymethylmethacrylate, gold, alloy, platinum, titanium, and stainless steel.

7.-28. (canceled)
